(19) United States
(12) Patent Application Publication
(10) Pub. No.: US 2014/0147503 A1
(43) Pub. Date: May 29, 2014

(54) Pharmaceutical Composition Comprising Deferasirox

(71) Applicants: Geena Malhotra, Mumbai (IN); Shrinivas Madhukar Purandare, Mumbai (IN)

(72) Inventors: Geena Malhotra, Mumbai (IN); Shrinivas Madhukar Purandare, Mumbai (IN)

(73) Assignee: CIPLA LIMITED, Mumbai (IN)

(21) Appl. No.: 13/825,471

(22) PCT Filed: Sep. 30, 2011
(86) PCT No.: PCT/GB11/01428

§ 371 Date: Apr. 19, 2013

(30) Foreign Application Priority Data
Oct. 1, 2010 (IN) 2750/MUM/2010

(51) Int. Cl. A61K 31/4196; A61K 45/06

(52) U.S. Cl. CPC A61K 31/4196; A61K 45/06
USPC 424/489; 548/268.6; 514/383; 514/199; 514/43; 514/20.5; 514/62; 424/85.4

(57) ABSTRACT

The present invention relates to a pharmaceutical composition comprising deferasirox, a process for preparing such pharmaceutical composition, and its use in the treatment of chronic iron overload. The pharmaceutical composition comprises nanosized deferasirox having improved surface area and solubility. It also relates to a method for treatment of chronic iron overload which comprises administering a pharmaceutical composition comprising nanosized deferasirox.

### FIELD OF INVENTION

The present invention relates to a pharmaceutical composition comprising an iron chelating agent, and more particularly, relates to a pharmaceutical composition comprising deferasirox or a pharmaceutically acceptable salt thereof, a process for preparing such pharmaceutical composition, and its use in the treatment of chronic iron overload.

### BACKGROUND AND PRIOR ART

One of the major obstacles to the development of highly potent pharmaceutical formulations is the poor water solubility of many drugs. Approximately 40% of potential drugs identified by pharmaceutical companies are poorly soluble in water, which greatly hinders their clinical use. Low water solubility limits the bioavailability and absorption of these agents.

Deferasirox has the chemical name 4-[3,5-bis(2-hydroxyphenyl)-[1,2,4]triazol-1-yl]enzoic acid, and is reported to have the following chemical structure.

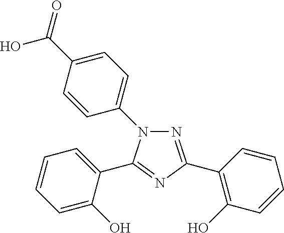 [SMILES: O=C(O)c1ccc(-n2nc(-c3ccccc3O)nc2-c2ccccc2O)cc1]

Deferasirox is an orally active iron chelator, and has been approved for the treatment of iron overload in transfusion dependent anemias (transfusion hemosiderosis), in particular thalassemia major, thalassemia intermediate and in sickle cell disease to reduce iron-related morbidity and mortality, in patients having an age of two years and older.

Chronic iron overload is a result of regular blood transfusions used in the treatment of several conditions including β-thalassemia, sickle cell disease and myelodysplastic syndromes.

Each unit of blood contains iron and as the human body has no physiological mechanism to actively excrete excess iron, repeated blood transfusions result in excessive accumulation of iron. This excess of iron deposited in body tissues can cause severe damage to organs such as liver, heart, endocrine organs. This may lead to many complications including cardiomyopathy, liver cirrhosis, diabetes mellitus and reduced life expectancy.

Deferasirox mobilizes tissue iron by forming soluble stable complexes that are then excreted in the feces. It is a tridentate iron chelator requiring two molecules of the drug to form a stable complex. Iron is chelated both from the reticuloendothelial cells (RE cells) as well as various parenchymal tissues. The chelated iron is cleared by the liver and excreted through the bile. It also has the ability to prevent the myocardial cell iron uptake by removing iron directly from myocardial cells.

Deferasirox is highly water-insoluble and is highly lipid-soluble, and is also observed to possess good permeability. According to the Bio-pharmaceutics Classification System (BCS), it has been classified as a Class II drug, implying that it is a poorly soluble, and a highly permeable drug. Though deferasirox is highly water-insoluble, whatever limited solubility it has, that too exhibits a high pH-dependent solubility. Though it is practically insoluble in lower pH, even at a pH of 6.8, it still remains insoluble, until the buffer strength is altered to get optimal dissolution profile.

Deferasirox being practically insoluble in aqueous media exhibits a generally poor dissolution profile and hence consequently poor bioavailability.

Several strategies and formulations have been employed to overcome these limitations of solubility and poor bioavailability. Although existing strategies such as complexing drugs with cyclodextrins, conjugation to dendrimers, salt formation of ionizable drugs and the use of co-solvents have been shown to improve drug solubility, solubilization methods that can improve the absorption of the drug are still highly desirable.

WO 2004035026 discloses a dispersible tablet of deferasirox wherein the active ingredient is present in an amount of from 5% to 40% by weight based on total weight of the tablet.

WO 2005097062 discloses a dispersible tablet of deferasirox wherein the active ingredient is present in an amount of from 42% to 65% by weight based on total weight of the tablet.

WO 2007045445 discloses a dispersible tablet of deferasirox or a pharmaceutically acceptable salt thereof present in an amount of from 42% to 65% by weight based on total weight of the tablet and at least one pharmaceutically acceptable excipient suitable for the preparation of dispersible tablets and to process for making said dispersible tablet.

WO 2009067557 discloses a process of preparing deferasirox formulations having sufficiently high dissolution rate and good bioavailability wherein said process comprises co-milling deferasirox with at least two pharmaceutically acceptable excipients in the absence of any solvent.

WO 2010035282 discloses oral pharmaceutical composition comprising deferasirox in the form of a dispersible tablet wherein the active ingredient has a mean particle size less than about 100 μm and is present in an amount greater than 66% by weight based on total weight of the tablet.

Deferasirox is commercially available as dispersible tablet (EXJADE®) for oral administration. EXJADE is supplied as a dispersible tablet containing 125 mg, 250 mg and 500 mg of deferasirox per tablet. This tablet is dispersed in a glass of water or any other suitable drink, and this resulting suspension is then administered to the patient.

Deferasirox is administered as a once daily oral iron chelator, which is prescribed as a dispersible tablet, i.e., a tablet which needs to be dispersed in an aqueous medium prior to administration.

Deferasirox is typically administered at an initial dose of about 20 mg/kg body weight, and the dose is adjusted up to a maximum of 30 mg/kg body weight.

Further, the recommended dosage of deferasirox is on the higher side in order to have a clinical benefit. Due to its high dosage, the overall tablet weight and its volume including its dimension makes it inconvenient to administer, in order to provide a pharmacologically active daily dosage amount of deferasirox.

Hence, there is a need for an oral dosage form having a high drug content which would be convenient for patient administration, which would exhibit acceptable dissolution and absorption which leads to better bioavailability.

### OBJECT OF THE INVENTION

The object of the present invention is to provide a pharmaceutical composition comprising nanosized deferasirox having improved surface area and solubility.

Another object of the present invention is to provide a process for preparing the pharmaceutical composition comprising nanosized deferasirox.

Yet another object of the present invention is to provide a method for treatment of chronic iron overload which comprises administering a pharmaceutical composition comprising nanosized deferasirox.

### SUMMARY OF THE INVENTION

According to one aspect of the present invention there is provided a composition comprising deferasirox in the form of particles, wherein substantially the particles have an average particle size of less than or equal to about 2000 nm.

According to another aspect of the present invention there is provided a pharmaceutical composition comprising deferasirox and at least one excipient.

According to another aspect of the present invention there is provided a pharmaceutical composition comprising deferasirox and a pharmaceutically acceptable carrier.

According to another aspect of the present invention there is provided a process for preparing a pharmaceutical composition, which process comprising the steps of: homogenizing deferasirox and at least one excipient to produce a homogenized dispersion of the deferasirox in the excipient; and milling said homogenized dispersion to produce a slurry of particles having an average particle size of less than or equal to about 2000 nm.

According to another aspect of the present invention there is provided the use of a composition according to present invention in the manufacture of a medicament for treating chronic iron overload.

According to another aspect of the present invention there is provided a method of treating chronic iron overload comprising administering a therapeutically effective amount of a composition having deferasirox according to the present invention to a patient in need thereof.

According to another aspect of the present invention there is provided a method of treating chronic iron overload comprising administering a therapeutically effective amount of deferasirox according to the present invention to a patient in need thereof.

According to one aspect of the present invention there is provided a pharmaceutical composition comprising deferasirox or pharmaceutically acceptable salt, solvate, derivatives, hydrate, enantiomer, polymorph, complex, or mixtures thereof.

According to another aspect of the present invention there is provided a pharmaceutical composition comprising deferasirox or pharmaceutically acceptable salt, solvate, derivatives, hydrate, enantiomer, polymorph, complex or mixtures thereof wherein the deferasirox is in the nanosize range.

According to yet another aspect of the present invention there is provided a process for preparing a pharmaceutical composition comprising deferasirox or pharmaceutically acceptable salt, solvate, derivatives, hydrate, enantiomer, polymorph, complex or mixtures thereof wherein the deferasirox is in the nanosize range.

According to a further aspect of the present invention there is provided a method of treatment of chronic iron overload using a pharmaceutical composition comprising deferasirox or pharmaceutically acceptable salt, solvate, derivatives, hydrate, enantiomer, polymorph, complex or mixtures thereof wherein deferasirox is in the nanosize range.

### DETAILED DESCRIPTION OF THE INVENTION

In iron chelation therapy, a chelating drug binds with free or “labile” iron in the blood and organs, which allows for removal of excess iron from the body. Thus if more of deferasirox is available for chelation, there will be better removal of excess iron from the body.

Also, where multiple transfusions are repeatedly needed and phlebotomy is not possible, the chelation therapy provides a means of controlling the iron overload. The bioavailability (the percentage of the drug absorbed compared to its initial dosage) is limited by insolubility. Dissolution rate is a function of the surface area of the particles and solubility. Dissolution rate is a direct function of total surface area for a dispersed phase.

The recommended dosage of deferasirox is on the higher side, i.e., an initial dose of about 20 mg/kg body weight, and this dose is adjusted up to a maximum of 30 mg/kg body weight. Further, deferasirox has been classified as a Class II drug which exhibits poor solubility.

Therefore, formulating a suitable formulation of deferasirox with desirable advantages such as, easy to manufacture as well as which possess advantages such as patient compliance is a challenge.

The inventors of the present invention have found that, the solubility properties of deferasirox were improved by using nanosized deferasirox and thus leading to better bioavailability of the drug.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 00, Number 00, March 2010].

The term “nanosizing” as used herein refers to the reduction of deferasirox particle size to the sub-micron range. By submicron range, this suitably means having an average particle size of less than or equal to about 2000 nm.

Nanosizing leads to increase in the exposure of surface area of deferasirox particles leading to an increase in the rate of dissolution.

The present invention thus provides a pharmaceutical composition comprising deferasirox wherein deferasirox is in the nanosize range.

The term “nanosize” as used herein refers to deferasirox particles having an average particle size of less than or equal to about 2000 nm, preferably less than or equal to about 1000 nm. The average particle size may, for example, be measured using laser based particle size analyzer.

Preferably, substantially all particles have a particle size of less than or equal to about 2000 nm, preferably less than or equal to about 1000 nm.

The term “particles” as used herein refers to individual particle of deferasirox, or particles of deferasirox or deferasirox granules or deferasirox compositions and/or mixtures thereof.

The average particle size of the deferasirox is preferably above 1 nanometre.

The term “Deferasirox” is used in broad sense to include not only “Deferasirox” per se but also their pharmaceutically acceptable salts, pharmaceutically acceptable solvates, pharmaceutically acceptable hydrates, pharmaceutically acceptable enantiomers, pharmaceutically acceptable esters, pharmaceutically acceptable derivatives, pharmaceutically acceptable polymorphs, pharmaceutically acceptable prodrugs, pharmaceutically acceptable complexes etc.

The nanoparticles of the present invention can be obtained by any of the process such as but not limited to milling, precipitation and homogenization.

According to one embodiment of the present invention, the process of milling comprises dispersing deferasirox particles in a liquid dispersion medium in which deferasirox is poorly soluble, followed by applying mechanical means in the presence of grinding media like milling pearls to reduce the particle size of deferasirox to the desired average particle size.

According to another embodiment of the present invention, the process of precipitation involves the formation of crystalline or semi-crystalline deferasirox nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nanocrystals are then formed by adding the organic mixture to an antisolvent like water in the presence of stabilizers such as Tween 80, Poloxamer 188 or lecithin. The choice of solvents and stabilizers and the mixing process are key factors to control the size and stability of the drug nanocrystals.

According to one another embodiment of the present invention, the process of homogenization involves passing a suspension of crystalline deferasirox and stabilizers through the narrow gap of a homogenizer at high pressure (for eg—500-2000 bar). The pressure creates powerful disruptive forces such as cavitation, collision and shearing, which disintegrate coarse particles to nanoparticles.

According to one more embodiment of the present invention, the process of spray-freeze drying involves the atomization of an aqueous deferasiorx solution into a spray chamber filled with a cryogenic liquid (liquid nitrogen) or halocarbon refrigerant such as chlorofluorocarbon or fluorocarbon. The water is removed by sublimation after the liquid droplets solidify.

According to a still another embodiment of the present invention, the process of supercritical fluid technology involves controlled crystallization of deferasiorx from dispersion in supercritical fluids, carbon dioxide.

According to another embodiment of the present invention, the process of double emulsion/solvent evaporation technique involves preparation of oil/water (o/w) emulsions with subsequent removal of the organic phase through evaporation. The emulsions are prepared by emulsifying an organic phase containing deferasirox, polymer and organic solvent in an aqueous solution containing emulsifier. The organic solvent diffuses out of the polymer phase and into the aqueous phase, and is then evaporated, forming deferasirox-loaded polymeric nanoparticles.

According to a further embodiment of the present invention, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of deferasirox ranging from 20 nm to more than 100 μm.

According to one further embodiment of the present invention, the process of thermal condensation involves use of capillary aerosol generator (CAG) to produce high concentration condensation submicron to micron sized aerosols from a deferasirox solution.

According to still further embodiment of the present invention, the process of ultrasonication may be used for nano-sizing deferasirox. The process of ultrasonication involves application of ultrasound during particle synthesis or precipitation, which leads to smaller particles of deferasirox and increased size uniformity.

According to another embodiment of the present invention, the nano-sized deferasirox may be prepared by spray drying. The process of spray drying involves supplying a feed solution at room temperature and pumping it through the nozzle where it is atomized by a nozzle gas. The atomized solution is then dried by preheated drying gas in a special chamber to remove moisture from the system, thus forming dry particles of deferasirox.

According to a preferred embodiment of the present invention, the nanomilled deferasirox may be obtained by nanomilling of deferasirox with at least one surface stabilizer, at least one viscosity building agent and at least one polymer.

The present invention thus provides a pharmaceutical composition comprising nanosized deferasirox particles, preferably in the form of granules. The granules may comprise at least one excipient. The excipient may comprise at least one of the following, but not limited to, at least one surface stabilizers, at least one viscosity building agent and at least one polymer and optionally other pharmaceutically acceptable carriers.

Surface stabilizer, according to the present inventions, means surfactants that are capable of stabilizing the increased surfaced charge of the nanomilled drug. Suitable amphoteric, non-ionic, cationic or anionic surfactants may be included as surface stabilizers in the pharmaceutical composition of the present invention.

According to the present invention, surfactants may comprise one or more, but not limited to Polysorbates, Sodium dodecyl sulfate (sodium lauryl sulfate), Lauryl dimethyl amine oxide, Docusate sodium, Cetyl trimethyl ammonium bromide (CTAB) Polyethoxylated alcohols, Polyoxyethylene sorbitan, Octoxynol, N,N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt etc.

Viscosity builders means, excipients that are capable of stabilizing the nanoparticles by increasing the viscosity of the composition and thus preventing physical interaction of nanoparticles under the operating conditions employed.

According to the present invention, viscosity builders, may comprise one or more, but not limited to derivatives of sugars, such as lactose, saccharose, hydrolyzed starch (maltodextrin) etc or mixtures thereof.

Polymers or polymers blends, according to the present invention, may comprise one or more hydrophilic polymers, but not limited to cellulose derivates like hydroxypropylcellulose, hydroxymethylcellulose, hydroxypropylmethylcellulose, methylcellulose polymers hydroxyethylcellulose, sodium carboxymethylcellulose, carboxymethylene and carboxymethyl hydroxyethylcellulose; acrylics like acrylic acid, acrylamide, and maleic anhydride polymers, acacia, gum tragacanth, locust bean gum, guar gum, or karaya gum, agar, pectin, carrageenan, gelatin, casein, zein and alginates, carboxypolymethylene, bentonite, magnesium aluminum silicate, polysaccharides, modified starch derivatives and copolymers.

The deferasirox composition having nanosized particles of the invention can be formulated into any suitable dosage form, including but not limited to liquid dispersions, gels, aerosols, ointments, creams, controlled release formulations, lyophilized formulations, tablets, capsules, delayed release formulations, extended release formulations, pulsatile release formulations, and mixed immediate release and controlled release formulations along with pharmaceutically acceptable carriers.

Solid oral dosage forms for administration include, but are not limited to, capsules, tablets, pills, powders, and granules. In such solid dosage forms, the active agent is admixed with at least one of the following carriers: (a) one or more inert excipients (or carriers) (b) fillers or extenders (c) binders (d) humectants (e) disintegrating agents (I) solution retarders (g) absorption accelerators (h) wetting agents (i) adsorbents and (j) lubricants. For capsules, tablets, and pills, the dosage forms may also comprise buffering agents.

The granules comprising nanosized deferasirox, according to the present invention, may either be encapsulated in capsules or be compressed to form tablets or may be provided as sachets or be provided as powders for reconstitution.

The solid dosage form, according to the present invention, may also optionally be coated. More preferably, the formulation may be seal coated and then film coated.

According to an embodiment of the present invention, pharmaceutical composition may be film coated with but not limited to Ready colour mix systems (such as Opadry colour mix systems) and Kollicoat® Protect.

According to the present invention, the seal coat comprises film forming polymeric materials, such as but not limited to, hydroxypropylmethylcellulose, hydroxypropylcellulose, polyvinylpyrrolidone, methylcellulose, carboxymethylcellulose, hypromellose, acacia, gelatin to increase adherence and coherence of the seal coat.

In one aspect of the present invention there is provided a pharmaceutical composition comprising deferasirox in the form of dispersible tablet, wherein deferasirox is in the nanosize range.

In another aspect of the present invention there is provided a pharmaceutical composition comprising deferasirox in the form of dispersible tablet, wherein deferasirox is in the nanosize range of less than or equal to about 2000 nm, preferably less than or equal to about 1000 nm.

The term “dispersible tablet” as used herein refers to a tablet which normally disperses in aqueous phase, e.g. in water, with or without external agitation.

Suitable carriers may be used for formulating the various dosage forms according to the present invention.

According to the present invention, pharmaceutically acceptable opacifier for use in the pharmaceutical composition of the present invention may comprise one or more, but is not limited to titanium dioxide.

According to the present invention, pharmaceutically acceptable diluents or fillers for use in the pharmaceutical composition of the present invention may comprise one or more, but not limited to lactose (for example, spray-dried lactose, α-lactose, β-lactose) lactose available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A 15C, Methocel A4M), hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and mixtures thereof.

According to the present invention, glidants, anti-adherents and lubricants may also be incorporated in the pharmaceutical composition of the present invention, which may comprise one or more, but not limited to stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumino metasilicate), sucrose ester of fatty acids, hydrogenated vegetable oils (for example, hydrogenated castor oil), or mixtures thereof.

According to the present invention, suitable binders may also be present in the pharmaceutical composition of the present invention, which may comprise one or more, but not limited to polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder.

According to the present invention, suitable disintegrants may also be present in the pharmaceutical composition of the present invention, which may comprise one or more, but not limited to hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and mixtures thereof.

Further, the pharmaceutical composition according to the present invention may further comprise at least one additional active ingredient such as but not limited to leukotriene, probenecid, indomethacin, penicillin G, ritonavir, indinavir, saquinavir, furosemide, methotrexate, sulfinpyrazone, interferon, ribavirin, viramidine, valopicitabine, aromatase inhibitor, antiestrogen, anti-androgen, gonadorelin agonist, topoisomerase I inhibitor, topoisomerase II inhibitor, microtubule active agent, alkylating agent, anti-neoplastic, anti-metabolite, platin compound, anti-angiogenic compound, cyclooxygenase inhibitor, bisphosphonate, heparanase inhibitor, telomerase inhibitor, protease inhibitor, matrix metalloproteinase inhibitor, proteasome inhibitor, somatostatin receptor antagonist, anti-leukemic compound, ribonucleotide reductase inhibitor, S-adenosylmethionine decarboxylase inhibitor; ACE inhibitor, antibiotics such as gentamicin, amikacin, tobramycin, ciprofloxacin, levofloxacin, ceftazidime, cefepime, cefpirome, piperacillin, ticarcillin, meropenem, imipenem, polymyxin B, colistin and aztreonam; cyclosporin A, cyclosporin G, rapamycin.

There is also provided a process for preparing the pharmaceutical composition of the present invention, which process comprises homogenizing deferasirox and at least one excipient to produce a homogenized dispersion of the deferasirox in the excipient; and milling said homogenized dispersion to produce a slurry of deferasirox particles having an average particle size of less than or equal to about 2000 nm.

According to one embodiment the pharmaceutical composition of the present invention, may be prepared by a process which comprises (a) preparing a dispersion of deferasirox with Docusate sodium, HPMC, sodium lauryl sulphate and sucrose in purified water under stirring conditions; (b) homogenizing the dispersion of step (a) and then nanomilling the homogenized dispersion; (c) adsorbing the nanomilled drug by spraying the nanomilled slurry on lactose monohydrate, microcrystalline cellulose and crospovidone mixture in fluidized bed granulator; (d) drying and blending the granules obtained in step (c). The granules may be lubricated and finally compressed into tablets. The tablets obtained may be seal coated and then film coated.

The present invention further provides a method for treating chronic iron overload which method comprises administering a therapeutically effective amount of a pharmaceutical composition according to the present invention.

Furthermore, the present invention provides pharmaceutical compositions comprising deferasirox for use in the treatment of chronic iron overload.

The following examples are for the purpose of illustration of the invention only and are not intended in any way to limit the scope of the present invention.

### Example 1

| Sr. No. | Ingredients | Qty mg/tablet |
|---|---|---|
| | Binder Solution | |
| 1. | Deferasirox | 500.00 |
| 2. | Docusate Sodium IP | 5.00 |
| 3. | Hydroxypropylmethylcellulose 3 cps IP | 100.00 |
| 4. | Sodium lauryl sulphate IP | 14.00 |
| 5. | Sucrose IP | 150.00 |
| 6. | Purified water IP | q.s |
| | Dry Mix | |
| 7. | Lactose Monohydrate(200 mesh) IP | 175.00 |
| 8. | Microcrystalline Cellulose IP (Avicel PH 101) | 152.00 |
| 9. | Crospovidone IP | 50.00 |
| | Lubrication | |
| 10. | Crospovidone IP | 36.00 |
| 11. | Magnesium Stearate IP | 6.00 |
| | Total | 1188.00 |
| | Seal Coating | |
| 12. | Hydroxypropylmethylcellulose 3 cps IP | 12.00 |
| 13. | Isopropyl Alcohol IP | q.s |
| 14. | Dichloromethane BP | q.s |
| | Total | 1200.00 |
| | Film Coating | |
| 15. | Opadry AMB White OY-B-28920 INH | 25.00 |
| 16. | Purified Water | q.s. |
| | Total | 1225.00 |

Process:
1. Docusate sodium, HPMC, sodium lauryl sulphate and sucrose were solubalized in water under stirring conditions.
2. Deferasirox was dispersed in the solution obtained in step (1).
3. Above dispersion was homogenized and then nanomilled.
4. Nanomilled drug slurry was adsorbed by spraying on lactose monohydrate, microcrystalline cellulose and crospovidone mixture in a fluidized bed granulator.
5. Granules obtained were sized and lubricated.
6. Lubricated granules were finally compressed into tablets.
7. The tablets obtained were seal coated and then film coated.

### Example 2

| Sr. No. | Ingredients | Qty mg/tablet |
|---|---|---|
| | Binder Solution | |
| 1. | Deferasirox | 500.00 |
| 2. | Docusate Sodium IP | 5.00 |
| 3. | Hydroxypropylmethylcellulose 3 cps IP | 100.00 |
| 4. | Sodium lauryl sulphate IP | 14.00 |
| 5. | Sucrose IP | 150.00 |
| 6. | Purified water IP | q.s |
| | Dry Mix | |
| 7. | Lactose Monohydrate(200 mesh) IP | 175.00 |
| 8. | Microcrystalline Cellulose IP (Avicel PH 101) | 152.00 |
| 9. | Crospovidone IP | 50.00 |
| | Lubrication | |
| 10. | Crospovidone IP | 36.00 |
| 11. | Magnesium Stearate IP | 6.00 |
| | Total | 1188.00 |
| | Seal Coating | |
| 12. | Hydroxypropylmethylcellulose 3 cps IP | 12.00 |
| 13. | Isopropyl Alcohol IP | q.s |
| 14. | Dichloromethane BP | q.s |
| | Total | 1200.00 |
| | Film Coating | |
| 15. | Kollicoat Protect | 15.00 |
| 16. | Talc IP | 60.25 |
| 17. | Titanium dioxide IP | 3.75 |
| 18. | Purified Water IP | q.s. |
| 19. | Isopropyl Alcohol IP | q.s. |
| | Total | 1225.00 |

Process:
1. Docusate sodium, HPMC, sodium lauryl sulphate and sucrose were solubalized in water under stirring conditions;
2. Deferasirox was dispersed in the solution obtained in step (1);
3. Above dispersion was homogenized and then nanomilled.
4. Nanomilled drug slurry was adsorbed by spraying on lactose monohydrate, microcrystalline cellulose and crospovidone mixture in a fluidized bed granulator;
5. Granules obtained were sized and lubricated;
6. Lubricated granules were finally compressed into tablets; and
7. The tablets obtained were seal coated and then film coated.

### Example 3

| Sr. No. | Ingredients | Qty mg/tablet |
|---|---|---|
| | Binder Solution | |
| 1. | Deferasirox | 250.00 |
| 2. | Docusate Sodium | 5.00 |
| 3. | Hydroxypropylmethylcellulose | 15.00 |
| 4. | Sodium lauryl sulphate | 13.80 |
| 5. | Sucrose | 25.00 |
| 6. | Purified water | q.s. |
| | Dry Mix | |
| 7. | Lactose Monohydrate (200 mesh) | 154.70 |
| 9. | Crospovidone | 50.00 |
| | Lubrication | |
| 10. | Silicified Microcrystalline Cellulose | 50.00 |
| 11. | Crospovidone | 40.00 |
| 12. | Croscarmellose Sodium | 20.00 |
| 13. | Magnesium Stearate | 1.50 |
| 14. | Total | 625.00 |

Process:
1. Docusate sodium, HPMC, sodium lauryl sulphate and sucrose were solubalized in water under stirring conditions;
2. Deferasirox was dispersed in the solution obtained in step (1);
3. Above dispersion was homogenized and then nanomilled;
4. Nanomilled drug slurry was adsorbed by spraying on lactose monohydrate, microcrystalline cellulose and crospovidone mixture in a fluidized bed granulator;
5. Granules obtained were sized and lubricated; and
6. Lubricated granules were finally compressed into tablets.

### Example 4

| Sr. No. | Ingredients | Qty mg/tablet |
|---|---|---|
| | Binder Solution | |
| 1. | Deferasirox | 500.00 |
| 2. | Docusate Sodium | 10.00 |
| 3. | Hydroxypropylmethylcellulose | 30.00 |
| 4. | Sodium lauryl sulphate | 27.6 |
| 5. | Sucrose | 50.00 |
| 6. | Purified water | q.s |
| | Dry Mix | |
| 7. | Lactose Monohydrate (200 mesh) | 309.40 |
| 9. | Crospovidone | 100.00 |
| | Lubrication | |
| 10. | Silicified Microcrystalline Cellulose | 100.00 |
| 11. | Crospovidone | 80.00 |
| 12. | Croscarmellose Sodium | 40.00 |
| 13. | Magnesium Stearate | 3.00 |
| | Total | 1250.00 |

Process:
1. Docusate sodium, HPMC, sodium lauryl sulphate and sucrose were solubalized in water under stirring conditions;
2. Deferasirox was dispersed in the solution obtained in step (1);
3. Above dispersion was homogenized and then nanomilled;
4. Nanomilled drug slurry was adsorbed by spraying on lactose monohydrate, microcrystalline cellulose and crospovidone mixture in a fluidized bed granulator;
5. Granules obtained were sized and lubricated; and
6. Lubricated granules were finally compressed into tablets.

### Example 5

| Sr. No. | Ingredients | Qty mg/tablet |
|---|---|---|
| | Binder Solution | |
| 1. | Deferasirox | 250.00 |
| 2. | Docusate Sodium | 5.00 |
| 3. | Polyvinylpyrrolidone | 15.00 |
| 4. | Sodium lauryl sulphate | 13.80 |
| 5. | Sucrose | 25.00 |
| 6. | Purified water | q.s. |
| | Dry Mix | |
| 7. | Lactose Monohydrate (200 mesh) | 154.70 |
| 9. | Crospovidone | 50.00 |
| | Lubrication | |
| 10. | Silicified Microcrystalline Cellulose | 50.00 |
| 11. | Crospovidone | 40.00 |
| 12. | Croscarmellose Sodium | 20.00 |
| 13. | Magnesium Stearate | 1.50 |
| | Total | 625.00 |

Process:
1. Docusate sodium, PVP, sodium lauryl sulphate and sucrose were solubalized in water under stirring conditions;
2. Deferasirox was dispersed in the solution obtained in step (1);
3. Above dispersion was homogenized and then nanomilled;
4. Nanomilled drug slurry was adsorbed by spraying on lactose monohydrate, microcrystalline cellulose and crospovidone mixture in a fluidized bed granulator;
5. Granules obtained were sized and lubricated; and
6. Lubricated granules were finally compressed into tablets.

### Example 6

| Sr. No. | Ingredients | Qty mg/tablet |
|---|---|---|
| | Binder Solution | |
| 1. | Deferasirox | 500.00 |
| 2. | Docusate Sodium | 10.00 |
| 3. | Polyvinylpyrrolidone | 30.00 |
| 4. | Sodium lauryl sulphate | 27.6 |
| 5. | Sucrose | 50.00 |
| 6. | Purified water | q.s |
| | Dry Mix | |
| 7. | Lactose Monohydrate (200 mesh) | 309.40 |
| 9. | Crospovidone | 100.00 |
| | Lubrication | |
| 10. | Silicified Microcrystalline Cellulose | 100.00 |
| 11. | Crospovidone | 80.00 |
| 12. | Croscarmellose Sodium | 40.00 |
| 13. | Magnesium Stearate | 3.00 |
| | Total | 1250.00 |

Process:
1. Docusate sodium, PVP, sodium lauryl sulphate and sucrose were solubalized in purified water under stirring conditions;
2. Deferasirox was dispersed in the solution obtained in step (1);
3. Above dispersion was homogenized and then nanomilled;
4. Nanomilled drug slurry was adsorbed by spraying on lactose monohydrate, microcrystalline cellulose and crospovidone mixture in a fluidized bed granulator;
5. Granules obtained were sized and lubricated; and
6. Lubricated granules were finally compressed into tablets.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical composition comprising deferasirox in the form of particles, wherein the particles have an average particle size of less than or equal to about 2000 nm.

2. A pharmaceutical composition according to claim 1, wherein the particles have an average particle size of less than or equal to about 1000 nm.

3. A pharmaceutical composition according to claim 1, comprising at least one excipient selected from the group comprising at least one or more of: a surface stabilizer, a viscosity building went and a polymer.

4. (canceled)

5. (canceled)

6. (canceled)

7. A pharmaceutical composition according to claim 3, wherein the surface stabilizer is a surfactant, which is amphoteric, non-ionic, cationic or anionic or combinations thereof.

8. (canceled)

9. A pharmaceutical composition according to claim 4, wherein the surfactant comprises one or more of polysorbates; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyltrimethylammonium bromide, polyoxyl 10 lauryl ether, brij, a bile salt; sodium deoxycholate; sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonates; a naphthalenesulphonate; an olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol esters; an anhydrosorbitol ester or an ethoxylated derivative thereof; a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; or mixtures thereof.

10. A pharmaceutical composition according to claim 4, wherein the surfactant is comprises one or more of: docusate sodium and/of sodium lauryl sulphate.

11. A pharmaceutical composition according to claim 3, wherein the viscosity building agent comprises lactose; sucrose; saccharose; a hydrolyzed starch, maltodextrin; or a mixture thereof.

12. (canceled)

13. A pharmaceutical composition according to claim 3, wherein the polymer comprises hydroxypropylcellulose; hydroxymethylcellulose; hydroxypropylmethylcellulose; a methylcellulose polymer; hydroxyethylcellulose; sodium carboxymethylcellulose; carboxymethylene hydroxyethylcellulose; carboxymethyl hydroxyethylcellulose; an acrylic polymer, acrylic acid, acrylamide, and maleic anhydride polymers and copolymers; or a mixture thereof.

14. (canceled)

15. A pharmaceutical composition according to claim 1, wherein substantially all the particles have an average particle size above 1 nm.

16. A pharmaceutical composition comprising a composition according to claim 1 and a pharmaceutically acceptable carrier, optionally, wherein the particles are adsorbed on a surface of the pharmaceutically acceptable carrier.

17. (canceled)

18. A pharmaceutical composition according to claim 16, wherein the pharmaceutically acceptable carrier comprises: one or more diluents or fillers; one or more binders; one or more lubricants; one or more glidants; one or more disintegrants; one or more preservatives; one or more humectants; one or more solution retarders; one or more absorption accelerators; one or more wetting agents; one or more adsorbents; one or more buffering agents; or a mixture thereof.

19. A pharmaceutical composition according to claim 16, formulated for oral administration.

20. (canceled)

21. A pharmaceutical composition according to claim 19, which is in a form of a tablet, optionally, wherein the tablet is a dispersible tablet.

22. (canceled)

23. A pharmaceutical composition according to claim 1 for use in treating chronic iron overload.

24. A process for preparing a pharmaceutical composition, which process comprises the steps of:
(1) homogenizing deferasirox and at least one excipient to produce a homogenized dispersion of the deferasirox; and
(2) milling said homogenized dispersion to produce a slurry of deferasirox particles having an average particle size of less than or equal to about 2000 nm.

25. A process according to claim 24, further comprising:
(i) adsorbing the milled slurry on a pharmaceutically acceptable carrier to form granules, which are optionally compressed to form tablets; or
(ii) forming the slurring into a liquid dispersions, gels or aerosols.

26. (canceled)

27. (canceled)

28. A process according to claim 24, wherein the excipient is comprises one or more of: at least one surface stabilizer, at least one viscosity building agent and at least one polymer;
wherein the surface stabilizer comprises a surfactant which is amphoteric, non-ionic, cationic or anionic, and combinations thereof;
wherein the surfactant comprises one or more of: polysorbates; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyltrimethylammonium bromide, polyoxyl 10 lauryl ether, brij, a bile salt, such as sodium deoxycholate or sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonates; a naphthalenesulphonate; and olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol esters; an anhydrosorbitol ester or an ethoxylated derivative thereof; a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; and mixtures thereof;
wherein the surfactant comprises one or more of: docusate sodium and sodium lauryl sulphate;
wherein the viscosity building agent comprises one or more of: lactose, sucrose, saccharose, a hydrolyzed starch, maltodextrin, and mixtures thereof; and,
wherein the polymer comprises one or more of: hydroxypropylcellulose; hydroxymethylcellulose; hydroxypropylmethylcellulose; a methylcellulose polymer; hydroxyethylcellulose; sodium carboxymethylcellulose; carboxymethylene hydroxyethylcellulose; carboxymethyl hydroxyethylcellulose; an acrylic polymer acrylic acid, acrylamide, maleic anhydride polymers and copolymers; and mixtures thereof.

29. (canceled)

30. A method of treating chronic iron overload comprising administering a therapeutically effective amount of a pharmaceutical composition according to claim 1 to a patient in need thereof.

31. A pharmaceutical composition according to claim 1 further comprising one or more active(s) selected from: leukotriene, probenecid, indomethacin, penicillin G, ritonavir, indinavir, saquinavir, furosemide, methotrexate, sulfinpyrazone, interferon, ribavirin, viramidine, valopicitabine, aromatase inhibitor, antiestrogen, anti-androgen, gonadorelin agonist, topoisomerase I inhibitor, topoisomerase II inhibitor, microtubule active agent, alkylating agent, anti-neoplastic, anti-metabolite, platin compound, anti-angiogenic compound, cyclooxygenase inhibitor, bisphosphonate, heparanase inhibitor, telomerase inhibitor, protease inhibitor, matrix metalloproteinase inhibitor, proteasome inhibitor, somatostatin receptor antagonist, anti-leukemic compound, ribonucleotide reductase inhibitor, S-adenosylmethionine decarboxylase inhibitor; ACE inhibitor, antibiotics, such as gentamicin, amikacin, tobramycin, ciprofloxacin, levofloxacin, ceftazidime, cefepime, cefpirome, piperacillin, ticarcillin, meropenem, imipenem, polymyxin B, colistin and aztreonam; cyclosporin A, cyclosporin G, rapamycin or their pharmaceutically acceptable salts, solvates, tautomers, derivatives, enantiomers, isomers, hydrates, prodrugs or polymorphs thereof.

32. (canceled)
